(19) United States
(12) Patent Application Publication
(10) Pub. No.: US 2008/0146889 A1
(43) Pub. Date: Jun. 19, 2008

(54) Method of monitoring human physiological parameters and safty conditions universally

(75) Inventors: Shuenn-Tsong Young, Taipei City (TW); Chung-Wang Lee, Hsinchu (TW); Chih-Cheng Lu, Taipei City (TW); Tsair Kao, Taipei City (TW); Woei-Chyn Chu, Taipei City (TW)

Correspondence Address: BACON & THOMAS, PLLC, 625 SLATERS LANE, FOURTH FLOOR, ALEXANDRIA, VA, 22314, US

(73) Assignee: National Yang-Ming University, Taipei City (TW)

(21) Appl. No.: 11/637,737

(22) Filed: Dec. 13, 2006

(51) Int. Cl. A61B 5/00

(52) U.S. Cl. 600/300

(57) ABSTRACT

In a method of monitoring human physiological parameters and safe condition universally, the method is applied to a monitoring apparatus worn at an examinee's body and includes the steps of: monitoring the examinee's current plurality of physiological parameters and plurality of movement information; analyzing the movement information to determine whether or not the examinee is in motion; analyzing the physiological parameters to determine whether or not each physiological parameter is in compliance with a normal physiological standard preinstalled in the monitoring apparatus if the examinee is determined not in motion, and also determining whether or not each physiological parameter is in compliance with a normal physiological standard preinstalled in the monitoring apparatus; and issuing a first precaution reporting signal to an identified recipient and sending out the first precaution reporting signal via a wireless transmission, if the physiological parameters are incompliance with the normal physiological standards.

### FIELD OF THE INVENTION

The present invention relates to a method of monitoring human physiological parameters and safty conditions universally, and more particularly to a method of determining whether or not an examinee's latest movement is abnormal while the examinee is in motion, or the examinee's physiological parameters are in regular patterns while the examinee is not in motion. If there is any abnormal movement information or irregular patterns of physiological parameters, a report will be issued through a wireless network to achieve the effect of timely monitoring the examinee, s safety conditions.

### BACKGROUND OF THE INVENTION

As the current medical science is well developed, the efficiency of medical and hygiene services is enhanced, and our living environment is improved, elderly people are healthier and more long-lived than ever. Further, the world's average fertility rate drops gradually, and the ageing population tends to increase continuously, and the social cost and other related issues becomes more and more serious. For instance, adults and children have to go to school or work and cannot take care of the elderly family members, and thus people worry about the way of monitoring the health and illness of their elderly family members as well as the quality of medical care. In view of these problems, manufacturers recognize the good business opportunities of geriatric nursing and remote home care and leverage the constant advancement of information technologies and the development of mobile communication network to connect a medial unit such as a nursing home, a senior center or a health care center at a remote district with insufficient medical resources via a wireless network. Therefore, service providers can provide mobile medical health services at a remote district and establish a remote patient condition monitoring information system for monitoring a patient's conditions, and such system can serve as an extension of a hospital ward. As a result, medical resources can be shared for looking after patients at remote sites over a long period of time and further enhancing the operating efficiency of medical treatments as well as a patient's survival rate. There are many applications similar to the foregoing example and applied for remote home care and remote medical services, and these applications not only solve the social problems caused by the ageing population of developed countries of the world, but also help each hospital or clinic to achieve the objectives of sharing resources and saving costs, as well as effectively promoting home care and medical services to remote districts having insufficient medical resources.

As various different vital and physiological detection technologies and data transmission technologies are well-developed and the network becomes popular, many scholars tried to establish a correlation between a daily home behavior model with diseases and physiological disabilities. A recent remote monitoring research on determining the health conditions of an elderly person reports that the health, independence, illness, aging or disability of the elderly actually goes through a transformation process, but such delicate sophisticated process is not observed easily by care service providers, doctors and even elderly individuals themselves. Therefore, the scholars tried to show that some simple monitoring methods including an elderly person's mobility, blood oxygen, blood pressure, respiration rate, heart rate and electrocardiogram and even the elderly person's sleeping mode can be used for predicting the change of the elderly person's functional health conditions, and converting various medial information obtained from the foregoing monitoring process and transmitting the information in an electronic form via a fast broadband telecommunication network and creating a remote home care or remote medical service software that integrates texts, data, figures, images, sounds, audio/video contents. Therefore, an appropriate, timely and cost-effective notice can be issued and processed in order to reduce the morbidity rate of elderly individuals and maintain an independent good quality of life for them.

Since the examinee may have substantial body movements under the monitoring action, these substantial body movements usually make the monitoring action unable to detect the examinee's respiration rate and heart rate accurately, because the monitoring actions adopt a testing measure in contact with different parts of the human body or use a different testing method. As a result, the incorrect data so obtained misleads the monitoring mechanism, and wastes the resources of national medical treatments and involves unnecessary alerting and follow-up handling processes. Therefore, finding a way of designing a method of monitoring human physiological parameters and safty conditions universally becomes an objective of the remote home care or remote health service and a subject for manufacturers or service providers to make improvements, so that when a patient feels unwell or gets hurt while moving around or tripping over something, the method can issue a notice accurately and the necessary follow-up handling mechanism can take over the situation timely.

### SUMMARY OF THE INVENTION

In view of the foregoing shortcomings of the prior art that cannot detect the examinee's respiration rate and heart rate accurately when the examinee is moving under the monitoring action and result in obtaining incorrect data and misleading the monitoring mechanism, the inventor of the present invention based on years of experience to conduct extensive researches and experiments, and finally developed and designed a method of monitoring human physiological parameters and safty conditions universally in accordance with the present invention, in hope of using the examinee's moving condition to determine whether or not the latest movement is abnormal while the examinee is in motion, or each physiological parameter is in a regular pattern while the examinee is not in motion. If there is any abnormal movement information or irregular pattern of physiological parameters, a report will be issued through a wireless network to achieve the effect of timely monitoring an examinee's safety conditions.

Therefore, it is a primary objective of the present invention to provide a method of monitoring human physiological parameters and safty conditions universally, and the method is applied to a monitoring apparatus worn on an examinee's body, and the monitoring apparatus receives a plurality of movement information and a plurality of physiological parameters of the examinee's body to continuously monitor whether or not the examinee's body is situated in a safe and sound condition, wherein these movement information are used for determining whether or not the examinee is in motion. If the examinee is not in motion, each physiological parameter is determined whether or not it is in compliance with a normal physiological standard preset in the monitoring apparatus. If the physiological parameters are not in compliance with the normal physiological standards, a first precaution reporting signal will be issued to a hospital, a clinic or a service provider by a wireless method via a gateway and compiles and monitors the examinee's current physiological conditions. Regardless of the examinee's location, a notice will be issued to inform a related hospital, clinic, or service provider, once if the examinee's health is determined abnormal.

Another objective of the present invention is to use the monitoring apparatus to analyze the examinee's movement information, if the monitoring apparatus determines that the examinee is in motion, and also determine whether or not the movement information is in compliance with a predetermined abnormal movement standard. If the monitoring apparatus determines that the movement information is in compliance with the abnormal movement standard, then the examinee will be determined as to be situated in an involuntary falling condition, and a second precaution reporting signal will be issued by a wireless method via a gateway. Regardless of the examinee's location of the examine, an identified hospital, clinic or service provider can locate the monitoring apparatus that receives the notice, if the examinee is situated at an involuntary falling condition.

To make it easier for our examiner to understand the objective, technical characteristics and effects of the present invention, preferred embodiments will be described with accompanying drawings as follows:

### BRIEF DESCRIPTION OF THE DRAWINGS

FIG. 1 is a schematic view of a system architecture of the present invention;

FIG. 2 is a flow chart of a movement sensor module that starts collecting an examinee's information in accordance with the present invention;

FIG. 3 is a flow chart of a physiological parameter sensor module that starts collecting an examinee's information in accordance with a preferred embodiment of the present invention; and

FIG. 4 is a flow chart of a monitoring apparatus that analyzes an examinee's involuntary falling condition in accordance with a preferred embodiment of the present invention.

### DETAILED DESCRIPTION OF THE PREFERRED EMBODIMENTS

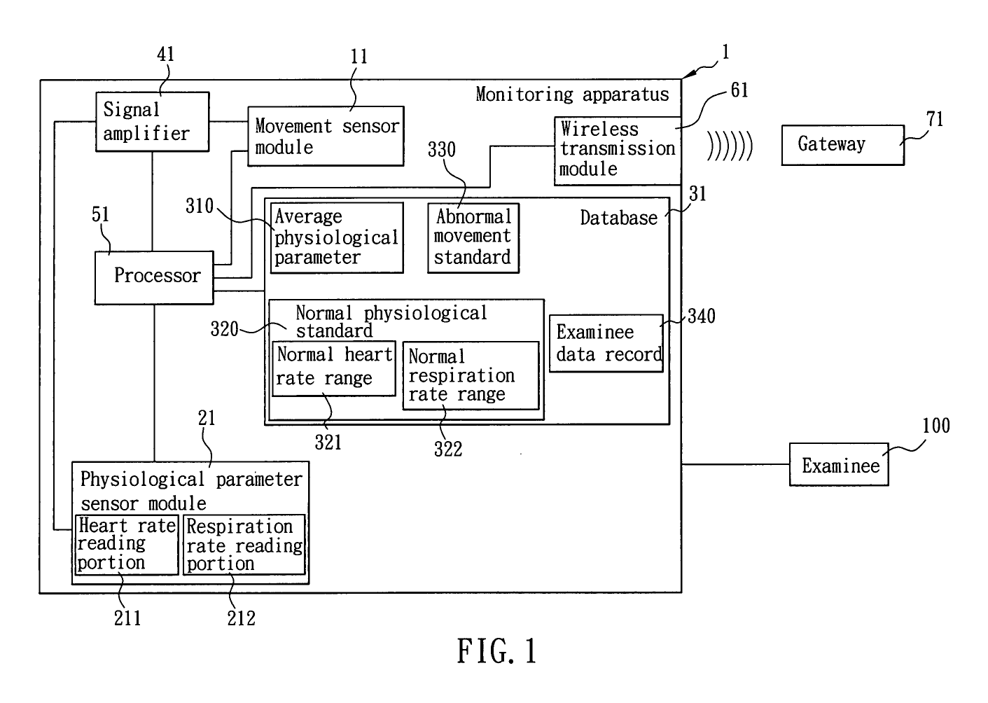

Referring to FIG. 1 for a method of monitoring human physiological parameters and safty conditions universally, the method is applied to a monitoring apparatus 1 that is worn on the body of an examinee 100, so that the monitoring apparatus 1 can receive a plurality of movement information (such as running or jumping) and a plurality of physiological parameters (such as respiration, heartbeat or body temperature) of the examinee 100 to continuously monitor whether the examinee is in a safe and sound condition, wherein the monitoring apparatus 1 analyzes the movement information first to determine whether or not the examinee 100 is in motion. If the examinee 100 is determined to be not in motion, then the monitoring apparatus 1 will further determine whether or not each physiological parameter is in compliance with a normal physiological standard 320 preset in the monitoring apparatus 1. If each physiological parameter is not in compliance with its corresponding normal physiological standard 320, then a first precaution reporting signal will be sent to an identified recipient such as a hospital, a clinic or a service provider (including a monitoring service provider or a nursing home) by a wireless method via a gateway and provided for them to compile and monitor the current physiological conditions of the examinee.

On the other hand, if the monitoring apparatus 1 determines that the examinee 100 is in motion, then the monitoring apparatus 1 will analyze the movement information to determine whether or not the examinee 100 is situated at a sudden abnormal movement (such as falling and lying on a floor) and no recovering movement back to its preceding state; if yes, then the monitoring apparatus 1 will deduce that the examinee 100 is situated at an involuntary fall state (such as falling down quickly and lying for a long time) and will issue a second precaution reporting signal to a hospital, a clinic or a service provider (such as a monitoring service provider or a nursing home) by a wireless method via a gateway for compiling and monitoring the current physiological conditions of the examinees.

Referring to FIG. 1 for a preferred embodiment of the present invention, the monitoring apparatus 1 comprises a movement sensor module 11 (such as a 3D direction sensor), a physiological parameter sensor module 21 (such as a thermometer or a sphygmomanometer), a database 31, and a signal amplifier 41 and a microprocessor 51 (such as a microcontroller) 51. The movement sensor module 11 is connected to the microprocessor 51 and the database 31, such that when the examinee 100 moves or leans forward, the swing produced by the examinee's body drives the monitoring apparatus 1 to receive a plurality of displacement data (such as displacement and angle of inclination), and the movement sensor module 11 computes the displacement data to obtain the displacement information (such as displacement acceleration and displacement direction) for accurately identify the difference between the examinee's falling down towards the ground quickly or the examinee's moving forward normally. The physiological parameter sensor module 21 is connected to the microprocessor 51, the signal amplifier 41 and the database 31, and comprises a heart rate reading portion 211 (such as an electrocardiography, ECG) and a respiration rate reading portion 212 (such as a micro motion detector sensor). The heart rate reading portion 211 can continue collecting the heartbeat amplitude of the examinee 100 within a predetermined time (such as 60 seconds) to compute a heart rate of the examinee 100 from the vibration produced by the heartbeat of the examinee 100. The respiration rate reading portion 212 can continue computing a respiration rate of the examinee 100 from the rise and fall of the respiration of the examinee 100 within a predetermined time (such as 60 seconds) to compute a respiration rate of the examinee 100, and the signal amplifier 41 can amplify the vibration produced by the heartbeat and the rise and fall of the respiration of the examinee 100 to avoid the situations of having an unduly weak data collection and unable to perform the counting and processing.

The database 31 and the microprocessor 51 are connected for recording a group of average physiological parameters 310, a group of normal physiological standards 320, a group of abnormal movement standards 330 and a plurality of examinee data records 340. The average physiological parameters 310 can use the monitoring apparatus 1 to obtain the data of the examinee 100 and update the data from time to time, and the normal physiological standards 320 include a normal heart rate range 321 (such as the heart rate falls in the range of 60˜100 times per minute) and a normal respiration rate range 322 (such as the respiration rate falls in the range of 12˜16 times per minute) based on the normal human physiological parameters, and such abnormal movement standards 330 are similar to the movement information (such as gravitational acceleration value, Gi or abnormal inclination, θb) produced by an involuntary fall of a human body, and the examinee data record 340 include an identification code (ID) of the monitoring apparatus 1, patient details of an examinee 100 and a contact information (such as a telephone number or an email address) of a plurality of related contact persons (such as a family member, a doctor or a medical hospital or a clinic).

The monitoring apparatus 1 further includes a wireless transmission module 61 for issuing a first or second precaution reporting signal to an external gateway 71 such that the gateway 71 will notice a remote hospital, a clinic or a service provider according to a predetermined mechanism if the monitoring apparatus 1 determines that there is a change to the most recently detected movement information or each physiological parameter is not complied. Although some prior arts have taught a monitoring apparatus 1 capable of roaming over an external network and having a reporting signal interactive system, these technologies are not related to the claims of the present invention, and thus will not be described here.

It is noteworthy to point out that when the examinee 100 starts using the monitoring apparatus 1, the monitoring apparatus 1 can record the following data in the database 31:

(1) The identification code of the monitoring apparatus 1 is the unique product number of the monitoring apparatus 1 and also an exclusive personal identification code of the examinee 100 after the examinee 100 has purchased the monitoring apparatus 100;

(2) The data including the patient details of the examinee 100, the contact information (such as a telephone number or an email address) of a related contact person (such as a family member, a doctor or a medical hospital or a clinic) correspond to the identification code of the monitoring apparatus 1, so that after the first and second precaution reporting signals are generated, the identification code of the monitoring apparatus 1 for receiving the first and second precaution reporting signals is used to identify the examinee 100 who uses the monitoring apparatus 1 and the related contact persons;

(3) When an examinee 100 wears the monitoring apparatus 1 for the first time, the monitoring apparatus 1 immediately obtains and records the physiological parameters (such as respiration, heartbeat or body temperature) of the examinee 100 as the average physiological parameter 310 of the database 31, and updates the average physiological parameter 310 from time to time.

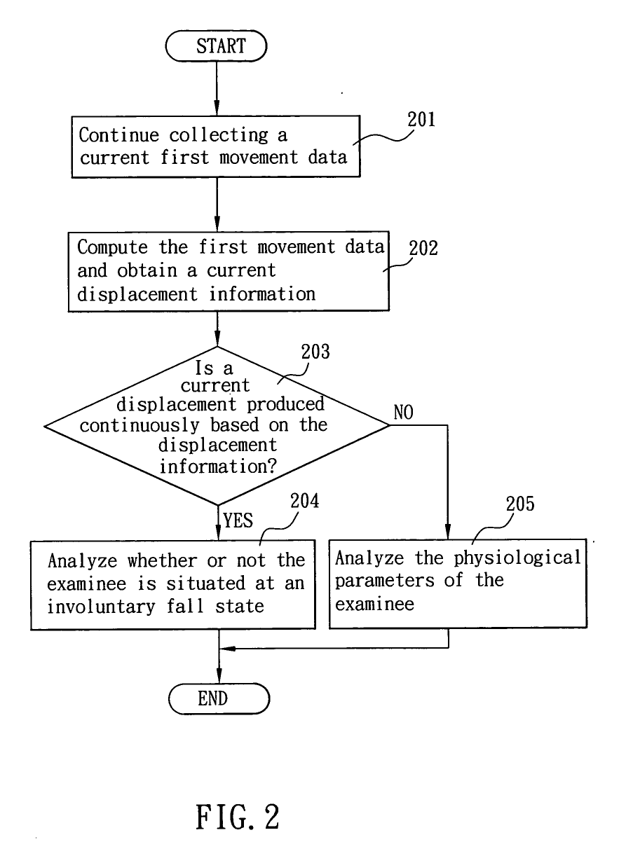

After the monitoring apparatus 1 is turned on as shown in FIG. 1 and the movement sensor module 11 starts collecting information of the examinee 100 as shown in FIG. 2, the microprocessor 51 will process the following procedure:

Step (201): The movement sensor module 11 continuously collects a plurality of first movement data (including displacement and angle of inclination) according to the current body movement of the examinee 100.

Step (202): The movement sensor module 11 obtains the current displacement information (such as moving direction and acceleration) of the examinee 100, after the first movement data is computed.

Step (203): This step determines whether or not the body of the examinee 100 continuously produces a displacement (such as determining whether or not the displacement produced at a specific time is greater than zero) based on the displacement information; if yes, then go to Step (204), or else go to Step (205);

Step (204): This step determines whether the examinee 100 is in motion and analyzes whether or not the examinee 100 is situated at an involuntary fall state.

Step (205): This step determines whether the examinee 100 is not in motion, and analyzes each physiological parameter of the examinee 100.

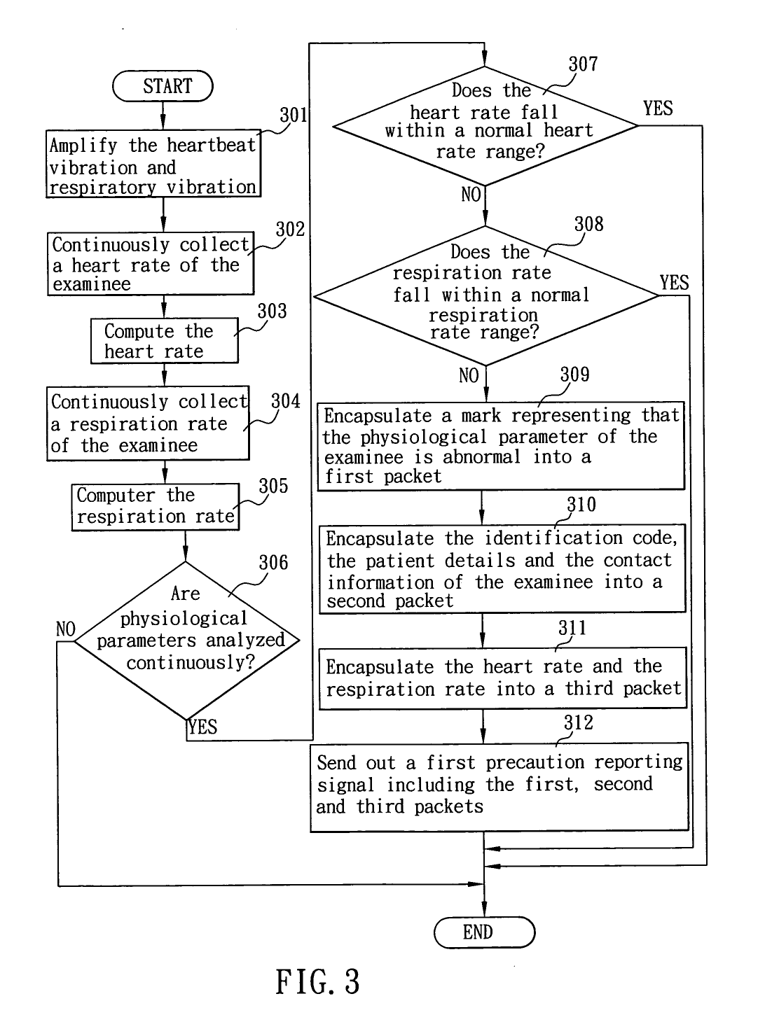

After the monitoring apparatus 1 is turned on, not only the movement sensor module 11 and the physiological parameter sensor module 21 start collecting information of the examinee 100, but the physiological parameter sensor module 21 also starts collecting information of the examinee 100 as shown in FIG. 3, and the microprocessor 51 will process the following procedure:

Step (301): The signal amplifier 41 amplifies the heartbeat vibration and respiratory vibration received by the examinee 100.

Step (302): The heartbeat vibration amplified by the signal amplifier 41 is provided for the heart rate reading portion 211 to continuously collect a heartbeat amplitude and a heart rate of the examinee 100.

Step (303): The heart rate reading portion 211 counts a heart rate of the examinee 100 within a predetermined time.

Step (304): The respiratory vibration amplified by the signal amplifier 41 is provided for the respiration rate reading portion 212 to continuously count the respiration rate of the examinee 100.

Step (305): The respiration rate reading portion 212 counts a respiration rate of the examinee 100 within a predetermined time.

Step (306): This step determines whether or not each physiological parameter of the examinee 100 is analyzed; if yes, then go to Step (307), or else end the procedure.

Step (307): This step reads the normal heart rate range 321 of the normal physiological standard 320 and determines whether or not the a rate computed by the heart rate reading portion 211 falls within the normal heart rate range 321; if yes, then skip this step and end the procedure, or else go to Step (308).

Step (308): This step reads the normal respiration rate range 322 of the normal physiological standard 320 and determines whether or not a respiration rate received by the respiration rate reading portion 212 falls within the normal respiration rate range 322; if yes, then skip this procedure and end this procedure, or else go to Step (309).

Step (309): This step encapsulates a mark representing that the physiological parameter of the examinee 100 is abnormal into a first packet.

Step (310): This step reads the examinee data record 340 and encapsulate the identification code of the monitoring apparatus 1, the patient details of the examinee 100 and the contact information of related contact persons into a second packet.

Step (311): This step encapsulates the heart rate and the respiration rate into a third packet.

Step (312): The wireless transmission module 61 sends out a first precaution reporting signal including the first, second and third packets.

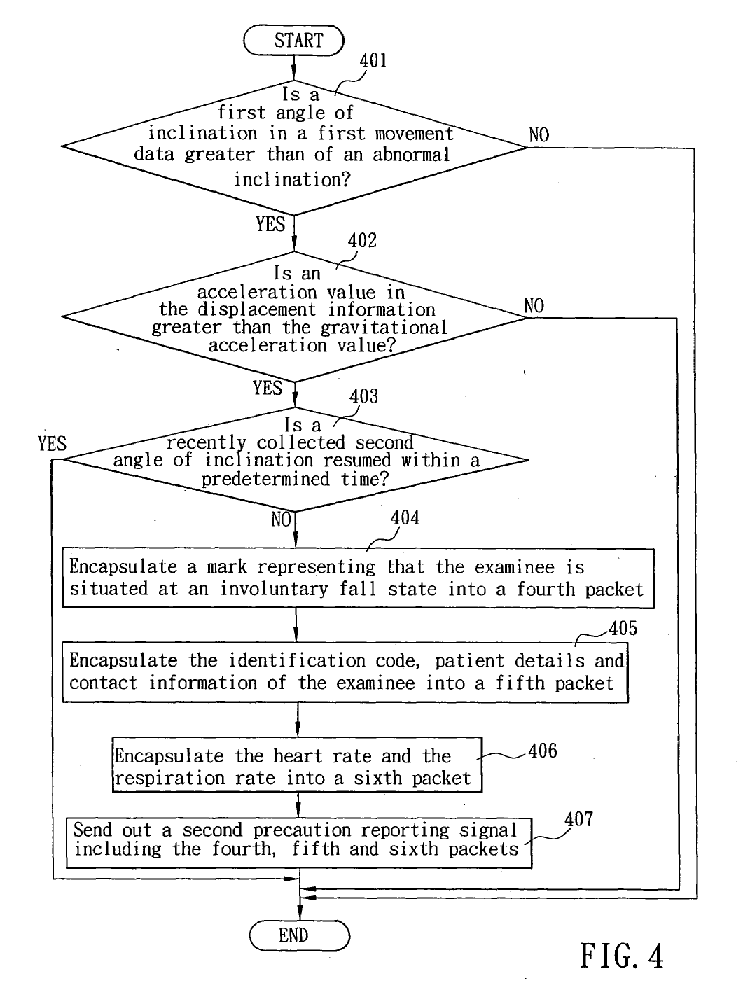

On the other hand, when the monitoring apparatus 1 analyzes whether or not the examinee 100 is situated at an involuntary fall state, the microprocessor 51 determines whether or not the displacement information (such as gravitational acceleration value Gi, or abnormal inclination θb) is in compliance with the abnormal movement standards 330 as shown in FIG. 1, and the microprocessor 51 will process the following procedure as shown in FIG. 4:

Step (401): This step determines whether or not a first angle of inclination (θa) in the current first movement data of the examinee 100 is greater than of an abnormal inclination (θb) set by the abnormal movement standards 330; if yes, then go to Step (402), or else skip this step and end the procedure.

Step (402): An abnormal movement standard 330 based on a gravitational acceleration value (Gi, which is equal to 9.80665 m/s²) is used for determining whether or not an acceleration value (ai) of the displacement information exceeds the gravitational acceleration value (Gi); if yes, then the examinee 100 will be determined as having a sudden abnormal movement (such as falling down quickly and lying for a long time) and go to Step (403), or else skip this step and end the procedure.

Step (403): A second displacement data of the body of the examinee 100 recently collected by movement sensor module 11 is used for determining whether or not a second angle of inclination (θc) of the second displacement is converted back to the angle of inclination (θa) within a predetermined time; if yes, then skip this step and end the procedure, or else go to Step (404).

Step (404): A mark representing that the examinee 100 is situated at an involuntary fall state” is encapsulated into a fourth packet.

Step (405): With the examinee data record 340, the identification code of the monitoring apparatus 1, the patient details of the examinee 100 and the contact information of related contact persons are encapsulated into a fifth packet.

Step (406): The heart rate and the respiration rate are encapsulated into a sixth packet.

Step (407): The wireless transmission module 61 sends out a second precaution reporting signal including the fourth, fifth and sixth packets.

Since the present invention uses the movement sensor module 11 to distinguish whether or not an examinee 100 is in motion and determine whether or not the latest movement information of the examinee 100 in motion is of a regular pattern or the examinee 100 is situated at an involuntary fall state (such as falling down quickly and lying for a long time). If the physiological parameter sensor module 21 determines whether or not a physiological parameter of the examinee 100 not in motion is abnormal, such that an abnormal movement information or physiological parameter is produced, a precaution reporting signal will be sent out via a wireless transmission to achieve the effect of timely monitoring the safe conditions of a human body, so that precautions can be reported accurately if a patient feels unease due to internal or external injuries while the patient is moving freely or having a trip over, and appropriate follow-up mechanisms can take over the situation timely.

While the invention herein disclosed has been described by means of specific embodiments, numerous modifications and variations could be made thereto by those skilled in the art without departing from the scope and spirit of the invention set forth in the claims.

What is claimed is:

1. A method of monitoring human physiological parameters and safty condition universally, which is applied to a monitoring apparatus worn at an examinee's body, comprising the steps of:
monitoring said examinee's current plurality of physiological parameters and plurality of movement information;
analyzing said movement information to determine whether or not said examinee is in motion;
analyzing said physiological parameters when said examinee is determined not in motion, and determining whether or not said each physiological parameter is in compliance with a normal physiological standard preinstalled in said monitoring apparatus; and
issuing a first precaution reporting signal to an identified recipient, when said physiological parameters are incompliance with said normal physiological standards, and sending out said first precaution reporting signal via a wireless transmission.

2. The method of claim 1, wherein when said monitoring apparatus monitors said examinee's current plurality of physiological parameters, said method further comprises the steps of:
continuously collecting said examinee's current heart rate by a heart rate reading portion of said monitoring apparatus;
counting said examinee's heart rate within a predetermined time by said heart rate reading portion;
continuously counting said examinee's current respiration rate by a respiration rate reading portion of said monitoring apparatus; and
counting said examinee's respiration rate within a predetermined time by said respiration rate reading portion.

3. The method of claim 2, wherein when said monitoring apparatus analyzes said movement information and determines whether or not said examinee is in motion, said method further comprises the steps of:
a movement sensor module of said monitoring apparatus continues collecting a plurality of first movement data produced by said examinee;
said movement sensor module computes said first movement data to obtain a current displacement information of said examinee;
said displacement information is used for determining whether or not said examinee continues producing a displacement; and
when said displacement data indicates that said examinee does not continue producing said displacement, then said examinee will be determined as not in motion, and said each physiological parameter of said examinee will be analyzed.

4. The method of claim 3, wherein when said monitoring apparatus analyzes said each physiological parameter of said examinee, said method further comprising the steps of:
reading a normal heart rate range of said normal physiological standards, and determining whether or not said heart rate computed by said heart rate reading portion falls within said normal heart rate range; and
reading a normal respiration rate range of said normal physiological standards when said heart rate does not fall within said normal heart rate range, and determining whether or not a respiration rate received by said respiration rate reading portion falls within said normal respiration rate range, and then continuing a process based on the coming step.

5. The method of claim 4, wherein when said monitoring apparatus determines that said physiological parameters are not in compliance with said normal physiological standards, then said monitoring apparatus will process a procedure comprising the steps of:
encapsulating a mark representing that said examinee physiological parameters are abnormal into a first packet;
reading an identification code preinstalled in said monitoring apparatus, an examinee's patient details, and encapsulating a plurality of contact information of a related contact person into a second packet;
encapsulating said heart rate and said respiration rate into a third packet; and
letting said wireless transmission module issue and send out a first precaution reporting signal including said first, second and third packets.

6. The method of claim 1, wherein when said examinee is determined as in motion, said method further comprising the steps of:
continuing to determine whether or not said movement information is in compliance with an abnormal movement standard preinstalled in said monitoring apparatus; and
defining said examinee as situating in an involuntary fall, when said movement information is in compliance with said abnormal movement standard, so as to issue a second precaution reporting signal to an identified recipient by a wireless transmission.

7. The method of claim 6, wherein after said monitoring apparatus is turned on and before said second precaution reporting signal is sent out, said method further comprises:
a movement sensor module of said monitoring apparatus continues collecting a plurality of first movement data produced by said examinee;
said movement sensor module computes said first movement data to obtain a current displacement information of said examinee;
said displacement information is used for determining whether or not said examinee continues producing a displacement; and
when said displacement data indicates that said examinee continues producing said displacement, then said examinee will be determined as in motion, and said examinee is analyzed to determine whether or not said examinee is situated in an involuntary fall state.

8. The method of claim 7, wherein when said monitoring apparatus analyzes whether or not said examinee is situated in an involuntary fall state, said method further comprises the steps of:
determining whether or not a first angle of inclination (θa) of said first movement data is greater than an abnormal angle of inclination (θb) set by said abnormal movement standards;
determining whether or not an acceleration value (ai) of said displacement information exceeds said gravitational acceleration value (Gi) based on a gravitational acceleration value (Gi) set by said abnormal movement standards, when said first angle of inclination (θa) is greater than said abnormal angle of inclination (θb);
determining whether or not one of said second angle of inclinations (θc) is resumed to said first angle of inclination (θa) in the latest updated second displacement collected by said movement sensor module within a predetermined time, when said acceleration value (ai) exceeds said gravitational acceleration value (Gi); and
defining said examinee as having an involuntary fall, when said second angle of inclination cannot be resumed to said first angle of inclination (θa) within a predetermined time, and then continuing a process based on the coming step.

9. The method of claim 8, wherein when said monitoring apparatus defines said examinee as having an involuntary fall, then said monitoring apparatus will process a procedure comprising the steps of:
encapsulating a mark representing that said examinee physiological parameters are abnormal into a fourth packet;
reading an identification code preinstalled in said monitoring apparatus, an examinee's patient details, and encapsulating a plurality of contact information of a related contact person into a fifth packet;
encapsulating said heart rate and said respiration rate into a sixth packet; and
letting said wireless transmission module issue and send out a second precaution reporting signal including said fourth, fifth and sixth packets.
